(19) United States
(12) Plant Patent Application Publication
(10) Pub. No.: US 2003/0028934 P1
(43) Pub. Date: Feb. 6, 2003

(54) Peach tree named 'GL V26'

(76) Inventors: Paul G. Glucina, Auckland (NZ); Michael T. Malone, Havelock North (NZ)

Correspondence Address: Michelle Bos, Stratton Ballew PLLC, 213 South 12th Avenue, Yakima, WA, 98902, US

(21) Appl. No.: 09/905,742

(22) Filed: Jul. 12, 2001

(51) Int. Cl. A01H 5/00

(52) U.S. Cl. PLT/195

(57) ABSTRACT

A new and distinct variety of peach tree (Prunus persica) named ‘GL V26’ is described. The new variety is a selection derived from the open pollinated seed of ‘Yumyeong’ (not patented), and is distinguishable from the parent variety by a dark red scarlet overcolour, and three weeks earlier harvest maturity than ‘Yumyeong.’ Its novelty consists of a unique combination of features that include late flowering, heavy and regular fruit production, large, very firm, white, crisp-textured, clingstone fruit. The fruit has a sweet, subacid flavor with very good eating quality.

### ORIGIN OF THE VARIETY

The present new and distinct variety was originated by Paul G. Glucina from an open pollinated population of seedlings derived from the variety ‘Yumyeong’ planted in 1986 at the HortResearch orchard in Kumeu, Auckland, New Zealand. A large group of open-pollinated seedlings were planted on their own roots, and under careful observation one seedling representing the present variety was selected and asexually propagated for further evaluation. The seedling was established at the HortResearch orchard at Havelock North, New Zealand and evaluated by Michael T. Malone. The selection was further propagated by asexual reproduction for the first time in 1989.

### ASEXUAL REPRODUCTION OF THE VARIETY

The new variety was budded onto ‘Golden Queen’ peach rootstock. The unique combination of characteristics and distinctive colour are true to the original seedling tree and are transmitted through succeeding asexual generations.

### SUMMARY OF THE VARIETY

The described variety of peach tree is of a medium to large size, vigorous, with a spreading growth habit. The new variety is a regular and productive bearer of medium to large, white flesh, clingstone fruit with a sweet sub-acid taste and very firm, crisp flesh. The fruit has very good flavour and eating quality. The firm flesh exhibits good storage and the skin has an attractive red colour over a cream background. The fruit ripens mid-season, approximately three weeks before the ‘Yumyeong’ parent variety.

### BRIEF DESCRIPTION OF THE DRAWINGS

The accompanying photographs show typical specimens of the foliage and the fruit of the new variety as depicted in colours as nearly true as is reasonably possible in a colour representation of this type. The photograph in FIG. 1 was taken shortly after harvest when the fruit was at eating maturity.

FIG. 1 shows the upper and lower leaf surfaces, and the exterior and sectional view of the fruit divided along the suture plane to show flesh colour, with the stone remaining in place; and,

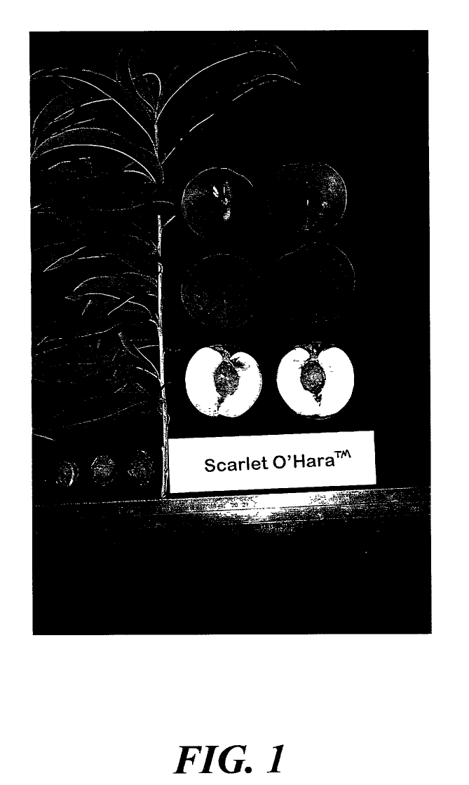

FIG. 2 shows typical fruit and leaves on the tree of the new variety.

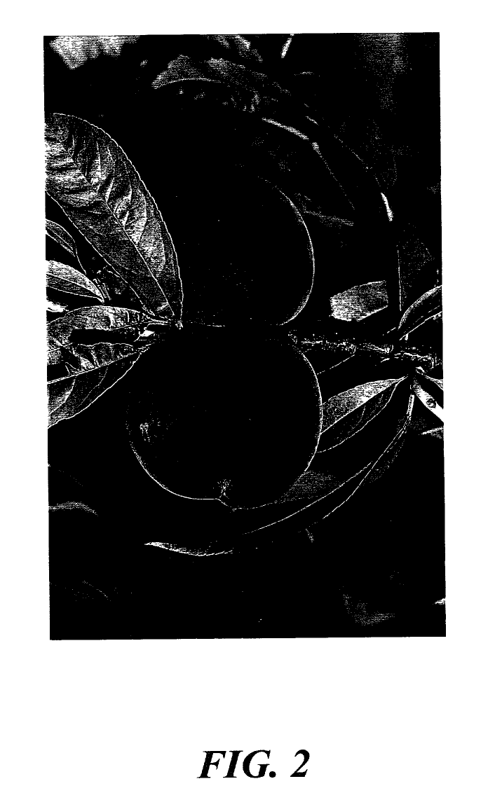

### DETAILED DESCRIPTION OF THE NEW VARIETY

The following is a detailed description of the new variety with colour terminology in accordance with The Royal Horticultural Society Colour Chart 1986 edition. The specimens described were grown in Havelock North, New Zealand. The observations were made in the 2001 season on trees which were four years old at the time, grown on ‘Golden Queen’ peach rootstock.

Tree:

Size.—Medium to large.

Vigour.—Medium.

Form.—Pruned to central leader, with a spreading growth habit.

Trunk:

Size.—Medium.

Texture.—Smooth.

Colour.—Grey/brown 199B.

Branches:

Size.—Medium.

Texture.—Smooth.

Lenticels.—Medium quantity, medium size.

Leaves:

Size.—Medium to large.

Average width.—42 mm.

Average length.—172 mm.

Shape of tip.—Acuminate.

Angle of tip.—Narrow acute.

Shape of base.—Attenuate.

Margin.—Crenate.

Thickness.—Medium.

Surface.—Smooth.

Petiole.—Medium length, medium thickness.

Glands.—Reniform, usually more than two, average 3-4 nectaries on fully developed leaf.

Color—upper surface.—Dark Green 141A.

Color—lower surface.—Green 146B.

Flowers:

Bud size.—Medium to large.

Bud length.—Medium.

Form.—Plump.

Flower size.—Medium to large.

Pollen.—Present.

Colour.—Medium pink.

Petal shape.—Round.

Pubescence of ovary.—Present.

Fruit:

Maturity for consumption.—Firm ripe.

First picking.—Mid-season.

Size.—Medium.

Average weight.—160 g.

Average diameter across suture line.—71 mm.

Average length.—63 mm.

Shape (ventral view).—Round oblate, symmetrical.

Shape of pistil end.—Weakly pointed.

Suture.—Medium to strong prominence, extends from base to apex.

Flesh:

Ripens.—Evenly.

Texture.—Firm crunchy, non-melting.

Fibres.—Non-fibrous.

Aroma.—Slight.

Average soluble solids.—12 Brix.

Average malic acid %.—0.48% with pH of 3.75.

Juice.—Moderate.

Colour.—White 155D; Anthocyanin coloration of flesh strongly expressed.

Stem:

Size.—Medium.

Skin:

Thickness.—Medium.

Adherence to flesh.—Medium to strong.

Pubescence density.—Medium.

Tendency to crack.—None.

Colour.—Red 47A, blending to a red-pink 49A, mottled pattern of over colour; Cream/yellow 11D ground colour of skin.

Stone:

Type.—Clingstone.

Size.—Medium.

Average stone length.—29 mm.

Average stone width.—22 mm.

Average stone thickness.—15 mm.

Average stone weight.—6.4 g.

Shape (lateral view).—Elliptic.

Surface.—Pits and grooves.

Tendency to split.—Low to medium, season variation.

Colour.—Yellow Brown 164A,B.

Use.—Local and export markets.

Storage.—Good; trials indicate 2-3 weeks storage without internal breakdown of flesh.

The present new variety of peach tree, its flowers, foliage and fruit herein described may vary slightly in detail due to climate, soil conditions and cultural practices under which the variety may be grown. The present description is that of the variety grown under the environmental conditions prevailing near Havelock North, Hawke's Bay, New Zealand.

We claim:

1. A new and distinct variety of peach tree named ‘GL V26,’ including all its parts and the fruit thereof, substantially as illustrated and described.
